(19) United States
(12) Patent Application Publication
(10) Pub. No.: US 2007/0016038 A1
(43) Pub. Date: Jan. 18, 2007

(54) Ultrasonic method to determine bone parameters

(76) Inventors: John E. Lynch, Williamsburg, VA (US); Joseph S. Heyman, Williamsburg, VA (US); Mark McKenna, Williamsburg, VA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/429,455

(22) Filed: May 4, 2006

Related U.S. Application Data
(60) Provisional application No. 60/678,554, filed on May 4, 2005.

(51) Int. Cl. A61B 8/00

(52) U.S. Cl. 600/438

(57) ABSTRACT

A method of measuring bone strength under dynamic loading is provided using an ultrasonic probe wave sensor to sense a low-frequency pump wave and an ultrasonic probe wave implemented to the bone. The bone is cyclically loaded with compressional and rarefactional pump waves, and probed with the probe wave that is timed according to the pump wave to determine the wave velocity of the probe wave. Bone strength is interpreted by measuring wave velocity changes during the pump wave cycles. Ultrasonic velocity derivatives are used to determine bone third-order (nonlinear) elastic constants that are linked to bone strength. High-resolution second-order (linear) elastic constants are provided through measurement of absolute phase velocity. A pulsed phase lock loop is locked at intervals as the probe wave phase is modulated over 360 degrees providing probe wave harmonic numbers that are correlated with the pump wave frequency to determine the probe wave velocity.

### CROSS-REFERENCE TO RELATED APPLICATIONS

This application is cross-referenced to and claims the benefit from U.S. Provisional Patent Application 60/678,554 filed May 4, 2005, which is hereby incorporated by reference.

### STATEMENT REGARDING FEDERALLY SPONSORED RESEARCH OR DEVELOPMENT

The present invention was supported in part by grant number NNC05CA44C from the NASA. The U.S. Government has certain rights in the invention.

### FIELD OF THE INVENTION

The invention relates generally to measuring bone strength. More particularly, the invention relates to measuring bone strength under dynamic loading.

### BACKGROUND

Variable and fixed frequency pulsed phase locked loops have been used to measure the phase shift caused by a delay path to a high degree of accuracy. Total phase changes through greater than 360 degrees have allowed for measurement of strain in bolts or other materials under static load.

Ultrasonic wave measurements using time intervals between like order echoes with respect to adjacent transmitted waves having an integral multiple of a period of a continuous oscillation wave has been used to obtain sound velocity.

A method and apparatus for measuring changes in intracranial pressure (ICP) utilizing the variation of the surface wave propagation parameters of the patient's skull to determine the change in ICP has been shown. The method uses a transmitted ultrasonic bulk compressional wave onto the surface of the skull at a predetermined angle with respect to the skull so as to produce a surface wave, receiving the surface wave at an angle with respect to the skull which is substantially the same as the predetermined angle and at a location that is a predetermined distance from where the ultrasonic bulk compressional wave was transmitted upon the skull, determining the retardation or advancement in phase of the received surface wave with respect to a reference phase, and processing the determined retardation or advancement in phase to determine circumferential expansion or contraction of the skull and utilizing the determined circumferential change to determine the change in intracranial pressure.

A measuring apparatus has used a fixed frequency oscillator to measure small changes in the phase velocity ultrasonic sound when a sample is exposed to environmental changes such as changes in pressure, temperature, etc. The apparatus automatically balanced electrical phase shifts against the acoustical phase shifts in order to obtain an accurate measurement of electrical phase shifts.

Noninvasive systems and methods have been used for the assessment of tissue properties by acquiring data relating to at least one aspect of intrinsic and/or induced tissue displacement, or associated biological responses. Data relating to tissue displacement and associated biological changes may be acquired by detecting acoustic properties of tissue using ultrasound interrogation pulses in a scatter or Doppler detection mode. Based on this data, tissue properties were assessed, characterized and monitored.

Changes in intracranial pressure have been measured dynamically and non-invasively by monitoring one or more cerebrospinal fluid pulsatile components. Pulsatile components such as systolic and diastolic blood pressures are partially transferred to the cerebrospinal fluid by way of blood vessels contained in the surrounding brain tissue and membrane. As intracranial pressure varies these cerebrospinal fluid pulsatile components also vary. Intracranial pressure has been dynamically measured by phase comparison of a reflected acoustic signal to a reference signal using a constant frequency pulsed phase-locked-loop ultrasonic device allows the pulsatile components to be monitored.

An ultrasonic therapeutic apparatus consisting of a therapeutic ultrasonic wave generating source driven by a driver circuit to generate therapeutic ultrasonic waves, an in vivo imaging probe so as to obtain a tissue tomographic image in the vicinity of the focus of the therapeutic ultrasonic waves is known. The imaging probe was used to receive echoes of the ultrasonic pulses emitted from therapeutic ultrasonic wave generating source. The driving conditions for the therapeutic ultrasonic wave generating source was adjusted on the basis of a received echo signal. The received echo signal contained information about actual intensity of the therapeutic ultrasonic waves within a living body.

A non-invasive system and method for inducing vibrations in a selected element of the human body and detecting the nature of responses for determining mechanical characteristics of the element is known. The method induced multiple-frequency vibrations in a selected element of the body by use of a driver; determining parameters of the vibration exerted on the body by the driver; sensed variations of a dimension of the element of the body over time, correlated the variations with frequency components of operation of the driver to determine corresponding frequency components of the variations, resolved the frequency components into components of vibration mode shape, and determined the mechanical characteristics of the element on the basis of the parameters of vibration exerted by the driver and of the components of vibration mode shape.

An ultrasound imaging system is known that utilized a short sinusoidal pulse burst for excitation, and performs coherent detection of the reflected signal. Density versus distance signal was reconstructed by integrating the coherently detected signal. The system included components to calculate and apply all necessary phase corrections.

The PPLL technique propagated a gated Radio Frequency (RF) acoustic wave into the sample. The acoustic wave propagated through the sample, reflecting from an interface and returning to the point of origin. The instrument sensed the pressure of the acoustic signal, gated the electrical signal from the sample that is produced by the reflected acoustic wave and samples the relative phase of the electrical signal by comparing its phase at an instant during each gating cycle with that of the continuously running voltage controlled oscillator (VCO) from which the initial driving signal was gated, A feedback loop is closed thus locking the frequency of the VCO to a fixed phase relationship with respect to the VCO. When the sample is loaded, strain plus sound velocity dependence on strain cause an acoustic phase shift producing a frequency shift in the VCO. The frequency shift divided by frequency F is linearly proportional to the applied load (for elastic loading). The device was used to accurately measure changes in strain independent of fastener friction.

Accordingly, there is a need to develop a method to determine bone parameters under dynamic loading in a clinical setting to overcome the current shortcomings in the art. It would be considered an advance in the art to provide a method of dynamically loading a bone in phase with a probing ultrasonic signal, to measure changes in sound velocity in the bone with respect to the phase of the loading force. This method of synchronous loading provides allows precise measurement of nonlinear elastic properties of bone without the application of potentially harmful loads, such as stressing by running or walking in a clinical setting. Furthermore, it is considered an advance in the art to provide a method for more accurately measuring the absolute speed of sound in bone when it is in an unloaded state, as an absolute measurement of sound velocity is linked to the linear elastic constant of bone, another important parameter in determining the structural soundness of bone.

### SUMMARY OF THE INVENTION

The present invention provides a new method of measuring bone parameters using ultrasonic velocity measurements. In one embodiment, bone strength is measured under dynamic loading by attaching a low-frequency pump wave transmitter to provide a low-frequency pump wave to the bone. Additionally, an ultrasonic probe wave transmitter to provide an ultrasonic probe wave is attached in the proximity of the bone. An ultrasonic probe wave sensor is attached to the proximity of the bone. The bone is dynamically loaded with the ultrasonic pump wave to periodically load the bone with compressional and rarefactional waves. The bone is probed during the dynamic loading with the ultrasonic probe wave that is timed according to the ultrasonic pump wave. The wave velocity of the ultrasonic probe wave is determined using the ultrasonic probe wave sensor. The bone strength is interpreted based on the detection of phase shifts in a reflected wave as the bone is loaded.

A method for measuring the absolute speed of sound with a commonly known method called the Pulsed-Phased-Locked-Loop (PPLL) is further provided. In this method, the PPLL is used as a basis but has been adapted to measure sound velocity with greater accuracy than previously possible. Measuring the absolute phase velocity of the probe wave is provided by inducing up to 360-degree phase changes in the probe wave through modulation of the PPLL control signal. By using PPLL with the probe wave, the PPLL is locked at a series of 360-degree phase changes to determine several harmonic numbers of the probe wave. These harmonic numbers are used to determine the probe wave velocity. In one embodiment, the frequency is varied such that the measured phase changes a full 360° and the PPLL is relocked, so that successive harmonics of the carrier frequency can be detected

The key advantages of the invention are providing dynamic loading of bone using noninvasive cyclical vibration while measuring nonlinear elastic constants of bone using probe waves. Nonlinear elastic constants are closely linked to bone strength, so that this method allows bone analysis in a clinical setting without the use of previous bone loading techniques that may cause harm or pain to the subject.

The invention also provides a method for more accurate determination of absolute sound velocity in bone, which is linked to the linear elastic constant of bone. Linear elastic constants have also been linked to bone strength, but the technique has large measurement uncertainties. More accurate measurements of sound velocity will reduce the error associated with this common measurement of bone quality.

### BRIEF DESCRIPTION OF THE FIGURES

The objectives and advantages of the present invention will be understood by reading the following detailed description in conjunction with the drawings, in which:

FIG. 1a shows experimental data for two samples of 4140 steel according to the present invention.

FIG. 1b shows pump beam measurements applied to a cortical bone sample from a turkey femur according to the present invention.

FIG. 2a shows timing circuitry according to the present invention.

FIG. 2b shows timing circuitry according to the present invention.

FIG. 3 shows the steps for measuring bone strength under dynamic loading according to the present invention.

FIG. 4 shows the steps for measuring the absolute phase velocity of the probe wave according to the present invention.

FIG. 5 shows a graph of a determined the speed of the probe wave phase velocity according to the present invention.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

Dynamic loading of bone can be achieved by applying external forces or activities including walking, stepping, standing, running, twisting, lifting, or flexing. In many situations it is desirable to assess bone strength without applying these external forces, for example in a clinical setting where these activities may not be practicable, or when a bone is thought to be frail or damaged and these activities may be harmful. A method for testing bone strength is presented that uses a high-power “pump” wave to cyclically load the bone, while the velocity of a low-power ultrasonic probe wave is measured as the pump wave cycles between compression and rarefaction.

The pump beam technique is a novel extension of a known class of measurements called ultrasonic derivative measurements, used to determine nonlinear elastic constants by measuring phase shifts with respect to a controlled load, either mechanical, thermal or magnetic. These phase shifts are proportional to changes in the speed of sound, which can be linked to a material's third-order elastic constant. This is a fundamental property of the material is closely linked to engineering properties such as strength. In this fashion, velocity derivatives can be used to nondestructively determine quantitatively the underlying properties of the material. It is known that higher order elastic constants are linked to engineering states/properties of applied stress, heat treatment, residual stress and fatigue. Using applied stress as an example, we explore the stress-strain equation
σ=k₂ε+k₃ε²+ . . . =k(ε) Equation 1
where σ is the stress, k₂ is the second order (linear) elastic constant and k₃ is the third order (nonlinear) elastic constant and ε is the strain. The ultrasonic velocity is related to the elastic constants (k(ε)) and the material density (ρ) by
V²=k(ε)/ρ≈(k₂+k₃ε)/ρ Equation 2

Taking a strain derivative of this equation reveals that
dV/dε=k₃/(2Vρ) Equation 3

Thus, the strain derivative of the velocity of sound is a parameter directly linked to the third-order elastic constant, a fundamental property of the material closely linked to nonlinear material behavior. In this fashion, we take velocity derivatives to determine quantitatively the underlying properties of the material nondestructively. A family of such measurements—including, for example, strain, pressure, heat and magnetic field derivatives—can be used to characterize engineering properties of materials such as strength and impact toughness.

In a typical derivative measurement, a change in an external parameter such as temperature or strain produces a corresponding change in ultrasonic phase. By performing a resonance measurement, the phase shift results in a change in resonance frequency, f. Thus,
df/f=j(dL/L) for a length “L” derivative Equation 4

Simply stated, the normalized change in frequency is directly related to a constant times the normalized parameter change. The constants in question are called third-order elastic constants and are affected by the material state. Accumulated damage alters some higher-order properties. For example, fatigue requires “material memory,” associated with micro-defect formation. These altered properties can be detected through velocity derivative measurements.

Strain derivatives have been used with great success to determine differences in strength and stiffness in materials as different as railroad rails and adhesive bonds, and provide insight into the strain state of a material when under load. Here, the applied load has been a relatively invasive static load, which may not be practical for clinical determination of bone strength. This work teaches a less-invasive method for applied a load to bone based on low-frequency mechanical waves, referred to as a pump beam. The current invention is a novel combination of synchronously timing the pump beam's compressional and rarefactional cycles with a probing beam used in combination with the PPLL.

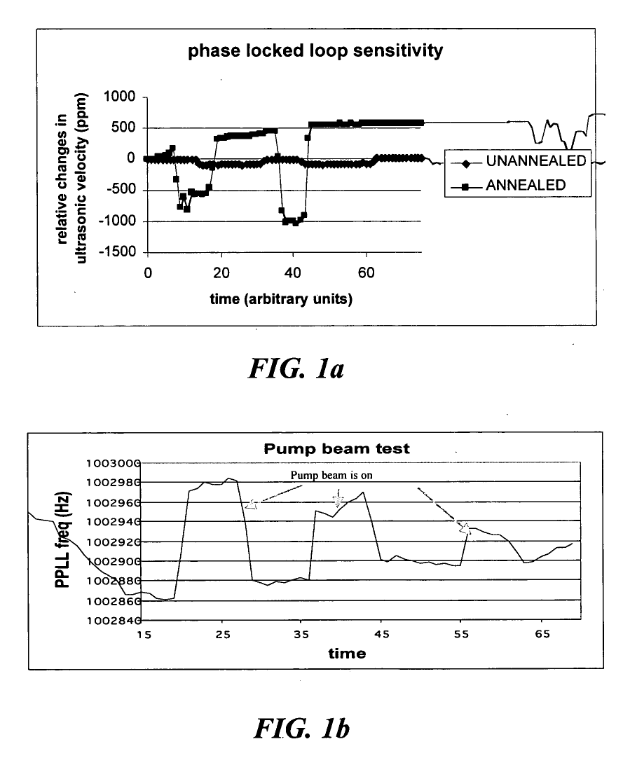

FIG. 1a shows experimental data for two samples of 4140 steel samples. The pump beam was applied for two arbitrary times for each of the samples. The annealed sample has much larger velocity changes, due to the dislocation density and lengths, indicating a larger nonlinear elastic constant.

FIG. 1b shows experimental data in which the pump beam measurement has been applied to pump beam results when the technique was applied to a cortical bone sample from a turkey femur.

The current method employs an ultrasonic measurement system, such as the pulsed phase locked loop, operating synchronously with a high-power, low-frequency pump wave to provide bone loading. Through the timing circuitry shown in FIG. 's 2a and 2b, this method will allow velocity measurements to be timed during a set number of positive half cycles of the pump wave, during which time the longitudinal wave is in compression, followed by a set number of a negative half cycles of the pump wave during which time the material is in rarefaction. Precisely timing the velocity measurement during each of the compressional and rarefactional cycles of the pump wave enables a method for detecting a material response under dynamic loading. The method in the current invention is considered an advancement in the art over traditional methods that average the response over the entire cycle, thus providing a measure of the material's nonlinear response to the pump wave only (in a nonlinear material, the behavior under compression is not the same as under rarefaction, so that an average over one cycle of the pump wave is not equal to zero).

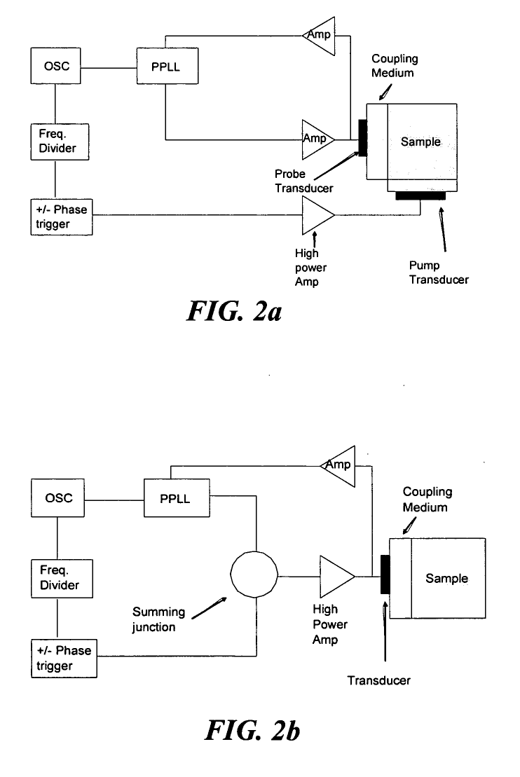

The timing circuitry in FIG. 2a is designed so that a master oscillator runs both the ultrasonic measuring device (PPLL) and the pump drive circuitry. A divide-by function times the pump drive-down by an integer value from the high-frequency ultrasonic signal.

Then a plus or minus trigger shifts the output to the amplifier by 180° depending on the whether the trigger is set to a rising or falling edge of the divided down signal.

An alternative embodiment is provided in FIG. 2b, in which a single transducer is employed and the pump and probe frequencies are combined through a mixer.

In this embodiment, the frequency of the pump signal should be low enough that the time for the pulse-echo pump signal to reflect off the material sample and return to the transducer is less than the duration of ½ cycle of the pump wave, or that tₚₑ=t_{1/2}. The pulse-echo time-of-flight, tₚₑ, is simply twice the distance between the bone and the transducer, divided by the speed of sound in the intervening material, plus the duration of the pulse-echo tone-burst, which equals the number of pulse cycles, n, divided by the tone-burst frequency or:
tₚₑ=2*d/v+n/f_{probe} Equation 5

The time of a half cycle of the pump frequency depends on the frequency of the probe wave f_{probe} the divide by value, m, as follows:
t_{1/2}=m/(2*f_{probe}) Equation 6

Setting tₚₑ=t_{1/2} and solving for the ratio f_{probe}/fₚᵤₘₚ in terms of f_{probe}, one obtains an expression for the minimum divide by value, mₘᵢₙ:
mₘᵢₙ=f_{probe}/[(4d)/v+2n/f_{probe}] Equation 7

The pump wave measurement is used to measure precise changes in the speed of sound as the pump wave switches from compression to rarefaction, where it is desirable to know how the speed of sound changes when loaded by the pump wave. The differential speed of sound with load is proportional to the measure of how much load is required to cause material break down, known as the third order elastic constant.

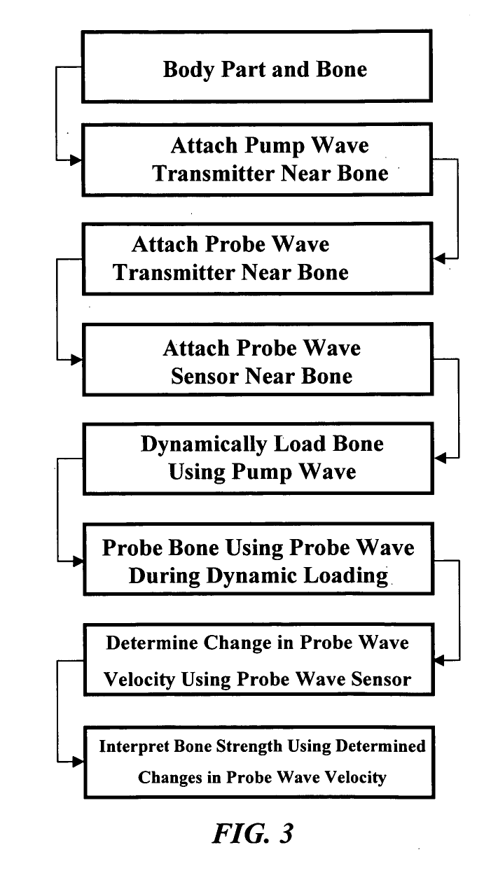

FIG. 3 depicts the steps for measuring bone strength under dynamic loading by providing a body or a body part enclosing a bone for measurement. The body part is equipped with at least one low-frequency pump wave transmitter in the proximity of the bone for producing a low-frequency pump wave to the bone. The body part is further equipped with at least one ultrasonic probe wave transmitter in the proximity of the bone for producing an ultrasonic probe wave to the bone. Additionally, the body part is equipped with at least one ultrasonic probe wave sensor in the proximity of the bone. The pump wave transmitter, probe wave transmitter and probe wave sensor may be combined to a single transducer or multiple transducers. The bone is then dynamically loaded with the ultrasonic pump wave to periodically load the bone with compressional and rarefactional waves. The bone is probed during this dynamic loading with the ultrasonic probe wave that is timed according to the ultrasonic pump wave, where probing can take place during compressional loading and during rarefactional loading. The probe wave velocity is determined using the probe wave sensor, where the boned strength can be interpreted using the determined wave velocity. In this method, the low-frequency pump wave has a frequency generally no more than one-half a pulse repetition frequency of the probe wave such that it contains an integer number cycles for each pulse of the probe wave. The ultrasonic probe wave frequency can range from 100 kHz to 5 MHz. Further, the pump wave generally has a power output sufficient to induce detectable changes in the speed of sound of the probe wave. The probe wave has a power output sufficient to produce detectable echo signals through the bone at a signal-to-noise ratio of approximately 20-40 dB.

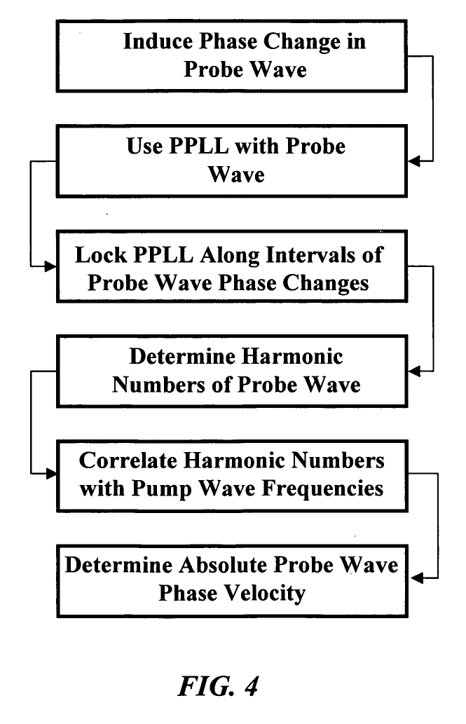

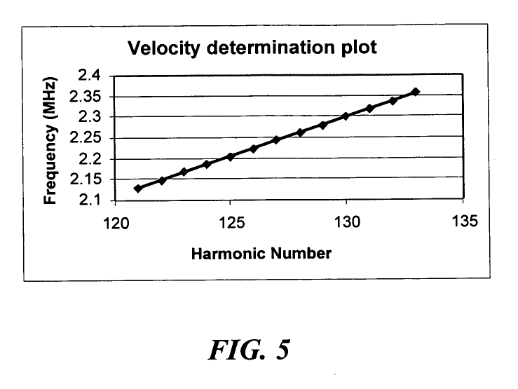

The absolute speed of sound is proportional to the measure of how much a material strains when tension is applied, known as the second order (linear) elastic constant. FIG. 4 depicts a further aspect of the invention showing the steps of a method for measuring the absolute phase velocity of the probe wave by varying the frequency of the pump wave to induce up to 360-degree phase changes in the probe wave. By using a pulsed phase lock loop (PPLL) with the probe wave, the PPLL is locked at intervals along the probe wave phase changes to determine several harmonic numbers of the probe wave. These harmonic numbers are correlated with the varying pump wave frequency to determine the probe wave velocity. In one embodiment, the frequency is varied such that the measured phase changes a full 360° and the PPLL is relocked, successive harmonics of the carrier frequency can be detected, as shown in the following equations:
fₘ₊₁−fₘ=v/2l(pulse-echo) Equation 8
fₘ₊₁−fₘ=v/l(through-transmission) Equation 9
where f is frequency, m and m+1 are two harmonics separated by 2π phase shift, v is the speed of sound and l is the distance between the transducer and reflector (pulse-echo mode) or the distance between two transducers (through-transmission mode). For a known “l,” a plot of frequency vs. harmonic number allows one to determine the velocity with great precision. FIG. 6 shows a graph of a determined the speed of probe wave phase velocity in a sample using the PPLL. In this example, the PPLL is unlocked and the output frequency is varied until the phase detector sweeps through a full 2π cycle. The sample position is advanced 1 cycle, and the PPLL is locked in quadrature. The frequency at the new position provides the next harmonic value in determining the speed of sound in a sample.

In this example, the velocity is determined from a statistical analysis of the family of lock points, mᵢ, extracting the enhanced accuracy from the number of points taken as well as averaging the frequency over a long period of time. For example, a 1 MHz frequency counted for 0.1 seconds has one-tenth the accuracy compared to counting for 10 seconds. By keeping all other parameters constant, such as temperature, strain, pressure, the velocity can be determined to high precision through these statistical procedures. The slope of the line in FIG. 6 is calculated using linear regression analysis, along with the standard error, where the slope of 18888 Hz gives fₘ₊₁−fₘ with a standard error of ±12 Hz. With a known Δl of 10 cm for example, this gives a speed of sound of 3777 m/s±2.4 m/s.

The present invention has now been described in accordance with exemplary embodiments, which are intended to be illustrative in all aspects, rather than restrictive. Thus, the present invention is capable of many variations in detailed implementation, which may be derived from the description contained herein by a person of ordinary skill in the art. For example the current invention may be used to evaluate strength of inhomogeneous medium in spacecraft, aircraft, automobiles or structures in situ, or prior to use or installation.

All such variations are considered to be within the scope and spirit of the present invention as defined by the following claims and their legal equivalents.

What is claimed is:

1) A method of measuring bone strength under dynamic loading, comprising:
a. providing a body or a body part enclosing said bone;
b. equipping at least one low-frequency pump wave transmitter providing a low-frequency pump wave to said body or said body part in proximity to said bone;
c. equipping at least one ultrasonic probe wave transmitter providing an ultrasonic probe wave to said body or said body part in proximity to said bone;
d. equipping at least one ultrasonic probe wave sensor to said body or said body part in proximity to said bone;
e. dynamically loading said bone with said ultrasonic pump wave to periodically load said bone with compressional and rarefactional waves;
f. probing said bone during said dynamic loading with said ultrasonic probe wave timed according to said ultrasonic pump wave;
g. determining changes in a wave velocity of said ultrasonic probe wave with said ultrasonic probe wave sensor as said pump wave cycles between said compressional wave and said rarefactional wave; and
h. interpreting said bone strength based on said determined changes in wave velocity.

2) The method according to claim 1, wherein said ultrasonic pump wave transmitter and said ultrasonic probe wave transmitter and said ultrasonic probe wave sensor are a single transducer.

3) The method according to claim 1, wherein said low-frequency pump wave has a frequency no more than one-half a pulse repetition frequency of said probe wave and contains an integer number cycles for each pulse of said probe wave.

4) The method according to claim 1, wherein said ultrasonic probe wave frequency ranges from 100 kHz to 5 MHz.

5) The method according to claim 1, wherein said pump wave has a power output sufficient to induce detectable changes in the speed of sound of said probe wave.

6) The method according to claim 1, wherein said probe wave has a power output sufficient to produce detectable echo signals through said bone at a signal-to-noise ratio of approximately 20-40 dB.

7) The method according to claim 1, wherein said determined wave velocity comprises a determined phase velocity of said probe wave.

8) The method according to claim 7, further comprising:
a. varying a control signal within said probe wave to induce up to 360 degree phase changes in said probe wave;
b. providing a pulsed phase lock loop to said probe wave;
c. locking said pulsed phase lock loop at intervals along said probe wave phase changes to determine a plurality of harmonic numbers of said probe wave; and
d. correlating said probe wave frequencies at said probe wave harmonic numbers to determine said probe wave velocity.
